(19) United States
(12) Patent Application Publication
(10) Pub. No.: US 2022/0057242 A1
(43) Pub. Date: Feb. 24, 2022

(54) Fluid Measurement Device

(71) Applicant: NIppon Telegraph and Telephone Corporation, Tokyo (JP)

(72) Inventor: Akio Tokura, Tokyo (JP)

(21) Appl. No.: 17/310,226

(22) PCT Filed: Jan. 14, 2020
(86) PCT No.: PCT/JP2020/000828

§ 371 Date: Jul. 27, 2021

(30) Foreign Application Priority Data
Jan. 28, 2019 (JP) 2019-011890

(51) Int. Cl. G01F 1/66; G01S 17/58; G01P 5/26; A61B 5/026

(52) U.S. Cl. CPC G01F 1/661; A61B 5/026; G01P 5/26; G01S 17/58

(57) ABSTRACT

First to N-th (N is an integer of three or more) sensor elements each including a light source unit and a light reception unit are arranged around a tube that allows a fluid containing scatterers to flow at equiangular intervals, and coherent light which is emitted from the light source unit of any one sensor element of the first to N-th sensor elements and is transmitted through the fluid flowing through the tube is received by the light reception unit of another predetermined sensor element of the first to N-th sensor elements. At this time, when a distance between the light source unit and the light reception unit of any one sensor element is d and an outer radius of the tube is r, a distance between any one sensor element and another predetermined sensor element is set to πd/2 or more and √3r or less.

### CROSS-REFERENCE TO RELATED APPLICATIONS

This patent application is a national phase filing under section 371 of PCT/JP2020/000828, filed Jan. 14, 2020, which claims the priority of Japanese patent application no. 2019-011890, filed Jan. 28, 2019, each of which is incorporated herein by reference in its entirety.

### TECHNICAL FIELD

The present disclosure relates to a fluid measurement device, and particularly to a fluid measurement device that measures a flow velocity, a flow rate, and the like of a fluid flowing through a flow path by using coherent light.

### BACKGROUND

A technology of measuring a flow velocity and a flow rate of a fluid flowing through a flow path has been widely used in industrial and medical fields. There are various types of fluid measurement devices that measure the flow velocity and the flow rate such as electromagnetic flowmeters, vortex flowmeters, coriolis flowmeters, ultrasonic flowmeters, laser flowmeters, and the like, and are used depending on applications. Of these fluid measurement devices, the laser flowmeter and the ultrasonic flowmeter are capable of measuring the flow velocity and the flow rate in a non-contact manner without coming into contact with the fluid flowing through the flow path, and thus, the laser flowmeter and the ultrasonic flowmeter are used in an application which requires hygiene, an application which does not allow the flowmeter to be inserted into an existing flow path, and the like.

Incidentally, although the ultrasonic flowmeter has high accuracy and is widely used, there is a problem in that an attempt of downsizing the flowmeter leads to high cost. In this regard, the laser flowmeter is easily downsized, and thus, a small flowmeter can be manufactured at low cost.

A laser Doppler flowmeter is used as the laser flowmeter (for example, see Patent Literature 1, Non-Patent Literature 1, and Non-Patent Literature 2). In this laser Doppler flowmeter, the flow path is irradiated with laser light which is one beam of coherent light or two beams of coherent light. When scatterers having a velocity contained in the fluid in the flow path pass through an irradiation region of the laser light, the laser light is scattered, and a frequency of the scattered light is subjected to Doppler shift. The frequency of the scattered light from a stationary object such as a flow path wall is not subjected to Doppler shift.

When the scattered light that is subjected to Doppler shift and the scattered light that is not subjected to Doppler shift are simultaneously received by a photodiode or the like and are converted into electrical signals, a beat signal is observed by performing heterodyne detection. When a frequency spectrum of the observed beat signal is calculated and a peak frequency is extracted, a moving velocity of the scatterers can be calculated. When a flow is a laminar flow, an average flow velocity and an average flow rate of the fluid flowing through the flow path are proportional to the moving velocity of the scatterers calculated by the above-described method, and thus, the average flow velocity and the average flow rate of the fluid can be calculated through calibration by multiplying the moving velocity by a proportional constant corresponding to the flow path.

Here, a configuration of a laser Doppler flowmeter of the related art will be described with reference to FIG. 7. FIG. 7 is a laser Doppler flowmeter that measures a flow rate of a tube through which a fluid flows (hereinafter, also referred to as a tube), and a tube 1 is made of a material having transmittance to light-source light (light from a light source unit 2). When the light-source light is, for example, visible light to near-infrared light, the tube 1 is made of, for example, vinyl chloride, and a cross section perpendicular to a flow path direction is, for example, a circle. The fluid includes a plurality of scatterers S.

A laser Doppler flowmeter 100 includes the light source unit 2, a light reception unit 3, a signal processing unit 4 that performs primary processing such as amplification or filtering of a received signal, and a calculation unit 5 that performs calculation processing based on a signal and the like, and a calculation result is sent to a result display unit 6 including a personal computer (PC), a display monitor, and the like for displaying a final measurement result.

The light source unit 2 is, for example, a semiconductor laser element (LD element) such as a surface-emitting laser, and is arranged around the tube 1 to irradiate the fluid with laser light. The light reception unit 3 is, for example, a photodiode (PD element), and receives and photoelectrically converts scattered light from the scatterers S in the fluid or scattered light from a stationary object such as a tube wall.

The light source unit 2 and the light reception unit 3 may be mounted in proximity to one board, or may be separate boards. In a method of the related art, the light source unit 2 and the light reception unit 3 are typically installed in proximity to each other to reduce a size of a sensor in many cases. In the present example, the light source unit 2 and the light reception unit 3 are mounted in proximity to a printed circuit board which is the signal processing unit 4. In the present example, the light source unit 2 and the light reception unit 3 are arranged in parallel with a tube axis J of the tube 1, that is, a direction of a flow of the fluid (see FIG. 8A), but may be arranged in a direction orthogonal to the direction of the flow of the fluid (see FIG. 8B).

A functional block diagram of the signal processing unit 4 and the calculation unit 5 illustrated in FIG. 7 is illustrated in FIG. 9. The signal processing unit 4 includes an amplifier 41 such as a transimpedance amplifier that amplifies a weak current signal from the light reception unit 3 and converts the current signal into a voltage signal, and a filter 42 such as a low-pass filter or a high-pass filter that extracts a desired band. The calculation unit 5 includes a data acquisition unit 51 such as an analog-to-digital conversion circuit (ADC circuit), and a calculation processing unit 52 that performs a fast Fourier transform (FFT) by using a calculator or the like. A secondary amplifier and a filter may be incorporated into the data acquisition unit 51 in front of the ADC circuit.

### CITATION LIST

### Patent Literature

Patent Literature 1: JP 57-059173 A

### Patent Literature

Non-Patent Literature 1: A. K. Jayanthy, et. al., “MEASURING BLOOD FLOW: TECHNIQUES AND APPLICATIONS—A REVIEW”, International Journal of Recent Research and Applied Studies, 6 (2011) pp. 203 to 216.
Non-Patent Literature 2: Armand Pruijmboom, et. al., “VCSEL-based miniature laser-Doppler interferometer”, Proc. of SPIE, Vol. 6908 (2008) pp. 69080I-1 to 69080I-7.

### SUMMARY

### Technical Problem

However, in such a laser Doppler flowmeter 100, the tube 1 has elasticity, and thus, the flow path is easy to bend. A flow velocity distribution is biased by the bending of the flow path and the like. This scene is schematically illustrated in FIGS. 10A and 10B.

FIG. 10A is a diagram illustrating a velocity distribution of a straight tube of which a flow path is not bent. A flow of the straight tube forms a uniform velocity distribution called a laminar flow under a condition in which the Reynolds number is equal to or less than a fixed value. A distribution in which the velocity is low in the vicinity of the tube wall which is easy to be influenced by viscosity and the velocity is high at a center of the tube is achieved. Such a distribution is achieved at any position of the tube, and thus, an average flow velocity and an average flow rate of the fluid flowing through the flow path are proportional to a moving velocity of the scatterers to be detected as described above. Thus, the average flow velocity and the average flow rate of the fluid can be calibrated and calculated by being multiplied by a proportional constant corresponding to the flow path.

Meanwhile, FIG. 10B is a diagram illustrating a velocity distribution of a tube of which a flow path is bent. In this case, due to the influence of bending, a distribution different from a laminar flow state found in the straight tube is achieved. Specifically, high-velocity components are biased outward of the bending (a side having a small curvature) due to the centrifugal force generated by a bent shape and a velocity of the fluid. A flow in a direction perpendicular to an axis of the tube, that is, in a radial direction, is formed by a pressure gradient due to this centrifugal force. Eventually, the fluid in the bent tube forms a velocity distribution with a spiral due to the combination of these flow components, and a non-uniform distribution is achieved. When the curvature of the tube fluctuates depending on each location, a velocity distribution in which the above-described effects are intricately entwined is formed. The moving velocity of the scatterers to be detected is a moving velocity of a local region, and thus, the curvature of the tube greatly fluctuates depending on a measurement position by reflecting the bias of the flow velocity distribution. Accordingly, it is very difficult to calculate the average flow velocity and the average flow rate of the fluid from the moving velocity.

Embodiments of the present disclosure have been made to solve such problems, and an object of the present disclosure is to provide a fluid measurement device capable of more accurately measuring an average flow velocity and an average flow rate of a fluid containing scatterers flowing through a tube made from an elastic body.

### Means for Solving the Problem

From the viewpoint of achieving such an object, embodiments of the present disclosure provide a fluid measurement device including first to N-th (N is an integer of three or more) sensor elements arranged around a tube that allows a fluid containing scatterers to flow, and each of the first to N-th sensor elements includes a light source unit configured to irradiate the fluid with coherent light and a light reception unit configured to receive and photoelectrically convert the coherent light, a signal processing unit configured to amplify and filter signals which are received and photoelectrically converted by the light reception units of the first to N-th sensor elements, and a calculation unit configured to convert the signals processed by the signal processing unit into digital signals, and calculate at least one of a flow velocity or a flow rate of the fluid based on the digital signals. The coherent light which is emitted from the light source unit of any one sensor element of the first to N-th sensor elements and is transmitted through the fluid flowing through the tube is received by the light reception unit of another predetermined sensor element of the first to N-th sensor elements, and when a distance between the light source unit and the light reception unit of the one sensor element is d and an outer radius of the tube is r, a distance between the one sensor element and the other predetermined sensor element is set to πd/2 or more and √3r or less.

In embodiments of the present disclosure, the coherent light which is emitted from the light source unit of any one sensor element (for example, first sensor element) of the first to N-th sensor elements (three or more sensor elements) and is transmitted through the fluid flowing through the tube is received by the light reception unit of another predetermined sensor element (for example, second sensor element) of which the distance from any one sensor element is πd/2 or more and √3r or less. Accordingly, forward-scattered light to be described below can be selectively detected, and the average flow velocity and the average flow rate of the fluid containing the scatterers can be more accurately measured even when the tube is made from an elastic body. The received signals of the first to N-th sensor elements are averaged, and thus, an influence of a change in a velocity distribution of the fluid caused by bending of the tube can be further reduced.

Note that, in the above description, by way of example, components in the drawings, which correspond to the components of the disclosure, are indicated by reference numerals in parentheses.

### Effects of Embodiments of the Invention

As described above, according to embodiments of the present disclosure, the first to N-th (N is an integer of 3 or more) sensor elements including the light source unit and the light reception unit are arranged around the tube, and the coherent light which is emitted from the light source unit of any one sensor element of the first to N-th sensor elements and is transmitted through the fluid flowing through the tube is received by the light reception unit of another predetermined sensor element of which the distance from the light source unit of any one sensor element is πd/2 or more and √3r or less. Thus, the forward-scattered light can be selectively detected, and the average flow velocity and the average flow rate of the fluid containing the scatterers flowing through the tube made from the elastic body can be more accurately measured. The received signals of the first to N-th sensor elements are averaged, and thus, an influence of a change in a velocity distribution of the fluid caused by bending of the tube can be further reduced.

### BRIEF DESCRIPTION OF THE DRAWINGS

FIG. 1 is a cross-sectional view illustrating an element arrangement in a fluid measurement device according to a first embodiment of the present disclosure.

FIG. 2A is a diagram illustrating an arrangement of a light source unit and a light reception unit in a sensor element.

FIG. 2B is a diagram illustrating the arrangement of the light source unit and the light reception unit in the sensor element.

FIG. 3A illustrates another example in which sensor elements are arranged around a tube at equiangular intervals.

FIG. 3B illustrates still another example in which the sensor elements are arranged around the tube at equiangular intervals.

FIG. 3C illustrates still another example in which the sensor elements are arranged around the tube at equiangular intervals.

FIG. 4A illustrates an example in which light rays from light source units of a plurality of sensor elements are received by a light reception unit of one sensor element.

FIG. 4B illustrates an example in which the light rays from light source units of the plurality of sensor elements are received by the light reception unit of one sensor element.

FIG. 4C illustrates an example in which the light rays from light source units of the plurality of sensor elements are received by the light reception unit of one sensor element.

FIG. 4D illustrates an example in which the light rays from light source units of the plurality of sensor elements are received by the light reception unit of one sensor element.

FIG. 5 is a cross-sectional view illustrating an element arrangement in a fluid measurement device according to a second embodiment of the present disclosure.

FIG. 6 is a diagram illustrating an example in which back-scattered light is received by using a light reception unit in the identical sensor element.

FIG. 7 is a diagram illustrating a configuration of a fluid measurement device of the related art.

FIG. 8A is a diagram illustrating an arrangement of light source units and light reception units in the fluid measurement device of the related art.

FIG. 8B illustrates the arrangement of the light source units and the light reception units in the fluid measurement device of the related art.

FIG. 9 is a functional block diagram of a signal processing unit and a calculation unit.

FIG. 10A is a diagram illustrating a velocity distribution in a straight tube (laminar flow state).

FIG. 10B illustrates a velocity distribution in a bent tube (non-laminar flow state).

FIG. 11 is a diagram illustrating a distance d between a light source unit and a light reception unit in an integrated sensor.

### DETAILED DESCRIPTION OF ILLUSTRATIVE EMBODIMENTS

Hereinafter, embodiments of the present disclosure will be described in detail. First, an outline of the present disclosure will be described before the embodiments are described.

### Outline of Disclosure

As described above, the reason why it is extremely difficult to calculate the average flow velocity and the average flow rate of the fluid from the moving velocity when the tube is made from the elastic body or the like and the tube may be bent is because velocity information of the scatterers to be detected is obtained from a local region.

Thus, in a situation in which the velocity distribution is not uniform such that the velocity distribution is changing depending on the position of the tube, a position at which a sensor is to be arranged or a bent state changes, and thus, values to be detected fluctuate. One method for solving this problem is to average these values by enlarging a region in which the velocity information of the scatterers to be detected is obtained. Thus, it is necessary to separate the light source unit and the light reception unit from each other such that scattered light generated from a wide range can be received.

However, an intensity of the scattered light is weak, and the light is scattered during repeated multiple scattering by separating the light source unit and the light reception unit from each other. Thus, there is a possibility that a light intensity becomes weak enough to be difficult to be detected. The scattered light is attenuated by absorption in a medium in which the light is absorbed.

From the viewpoints of solving such attenuation, it is necessary to detect the scattered light in a direction of a stronger scattering. That is, as in the related art, when the distance between the light source unit and the light reception unit is close, the light from the light source unit scattered backward of the scatterers (hereinafter, referred to as “back-scattered light”)) is necessarily received. However, in the fluid measurement device according to the present embodiment, this problem is solved by receiving the light from the light source unit scattered forward of the scatterers (hereinafter, referred to as “forward-scattered light”) instead. The “forward-scattered light” advances along an optical path through which the fluid is transmitted (passes), and thus, the forward-scattered light is included in “transmitted light” to be described below.

In blood to be often measured by the flowmeter, a size of a red blood cell (particle size) which is the scatterer is approximately identical to a wavelength used for measurement, and the scattering in this case is referred to as “Mie scattering”. In this type of scattering, an intensity of the forward-scattered light is about 10 times stronger than an intensity of the back-scattered light, and an attenuation amount of the light caused by separating the light source unit and the light reception unit from each other can be compensated for.

Accordingly, the light source unit and the light reception unit may have a “transmitted light detection arrangement” in which transmitted light from a light source (light transmitted through the fluid flowing through the tube (including forward-scattered light)) is received, and thus, the forward-scattered light can be selectively detected. This arrangement has an effect that information related to a concentration of the scatterers can also be obtained from the attenuation amount of the transmitted light caused by absorption and scattering by the scatterers to detect the transmitted light.

The light source unit and the light reception unit can be arranged as independent elements. However, data at various positions can be acquired by arranging a plurality of sensor elements (hereinafter, also referred to as “integrated sensor elements”) in which the light source unit and the light reception unit are provided in proximity to one board around the tube. Accordingly, the number of data itself increases, and thus, there is an advantage that measurement accuracy is improved.

Meanwhile, when the light source unit and the light reception unit have the “transmitted light detection arrangement”, the distance between the light source unit and the light reception unit is an important factor. As the optical path becomes long, the scattered light generated from a wider range becomes easy to be received. Thus, an averaging effect of the velocity distribution may be increased. However, while the intensity of the forward-scattered light is strong, as the optical path, that is, a transmission distance becomes long, diffusion attenuation and absorption attenuation caused by multiple scattering become easy to be caused even in the forward-scattered light. As a result, the intensity of the received signal decreases.

As verification for verifying the effect of the intensity of the received signal and averaging, when four sensors each including, for example, the light source unit and the light reception unit are installed around the tube at equiangular intervals (every 900), a fluid having a dilute concentration is measured in an arrangement of the light source unit and the light reception unit in which a diameter of the tube is connected (hereinafter, this arrangement is referred to as a “complete transmission arrangement”). At this time, it is experimentally confirmed for the first time that an influence of the bending of the tube is less by receiving the transmitted light (adjacent sensor arrangement) in the sensors positioned adjacent to each other, measuring the fluid, and averaging four sensor signals compared to a case where four sensor signals are averaged.

With such verification, to effectively reduce the influence of the bending of the tube, it is confirmed that the distance between the light source unit and the light reception unit in the measurement needs to be shorter than the complete transmission arrangement and an optimal number of sensor elements is three or more when the plurality of integrated sensor elements is used. It can be seen that an optimal distance between the light source unit and the light reception unit in the measurement is preferably equal to or less than a distance Lth between the sensor elements adjacent to each other when three integrated sensor elements are arranged at equiangular intervals. In this case, the distance Lth between the sensor elements adjacent to each other is √3r when an outer radius of the tube (a radius of an outer diameter) is r.

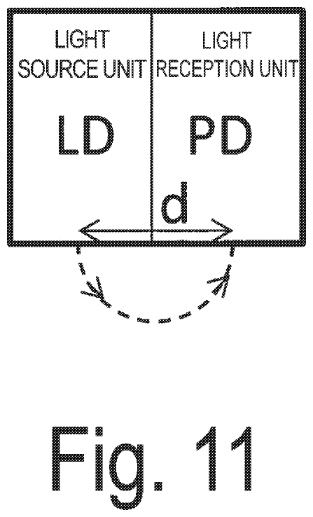

Embodiments of the present disclosure exhibit an effect compared to the method of the related art in which the back-scattered light is received by using the light source unit and the light reception unit of the integrated sensor when an optical path length in the measurement becomes long and the averaging effect of the velocity distribution increases. When it is assumed that the distance between the light source unit and the light reception unit in the integrated sensor is set to d (see FIG. 11), an average optical path length in the related art that receives the back-scattered light may be estimated as a length πd/2 of an arc. Thus, it is also confirmed from the verification that the optimal distance between the light source unit and the light reception unit in the measurement is πd/2 or more. Thus, although there is some variation caused by an arrangement relationship of the light source unit and the light reception unit within the sensor element by setting the distance between the sensor elements to πd/2 or more, the optical path length with which the present disclosure exhibits the effect is achieved regardless of the details of the arrangement relationship between the light source unit and the light reception unit. Accordingly, when the plurality of integrated sensor elements is used, the distance between the sensor element that irradiates the fluid with the coherent light and the sensor element that receives the coherent light is preferably πd/2 or more and √3r or less.

When the number of sensor elements to be arranged increases, the light is considered to be received by not only the adjacent sensor element but also by the adjacent sensor element of the adjacent sensor element (first adjacent element), that is, the sensor element (second adjacent element) adjacent with the adjacent sensor element interposed between these sensor elements adjacent to each other. However, in this case, it is needless to say that the distance between the light source unit and the light reception unit in the measurement is preferably equal to or less than the distance Lth between the sensor elements adjacent to each other described above. In this case, the number of optimal sensor elements is six or more.

When the integrated sensor elements are used, the back-scattered light can be received by using the light reception unit in the identical element, and thus, the flow velocity and the flow rate may be calculated by additionally using the received signal of the back-scattered light.

Processing such as standardization of the amount of light is easy to be performed in the measurement. Thus, the number of light source units received by each light reception unit is preferably one (each light reception unit does not receive light rays from two light source units), but light rays from a plurality of light source units may be simultaneously received when the measurement accuracy is not adversely influenced.

### First Embodiment

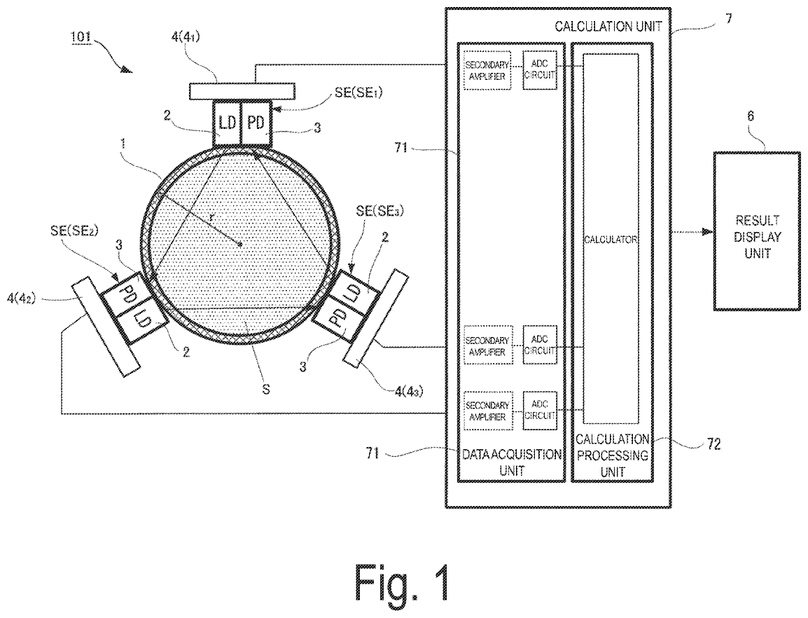

Hereinafter, a fluid measurement device according to a first embodiment of the present disclosure will be described with reference to the drawings. FIG. 1 is a cross-sectional view of an element arrangement in a fluid measurement device 101 according to the first embodiment. In this figure, components identical to the components described with reference to FIG. 7 are assigned the same reference signs, and descriptions will be omitted.

In the present embodiment, for example, vinyl chloride having an inner diameter 2r of 5.6 mm is used as a tube 1, and three integrated sensor elements SE in which a light source unit 2 and a light reception unit 3 are provided in proximity to one board are arranged around the tube 1 at equiangular intervals (intervals of 120°). In this case, distances between the light source units 2 and the light reception units 3 of the sensor elements SE adjacent to each other are equal between the three sensor elements SE.

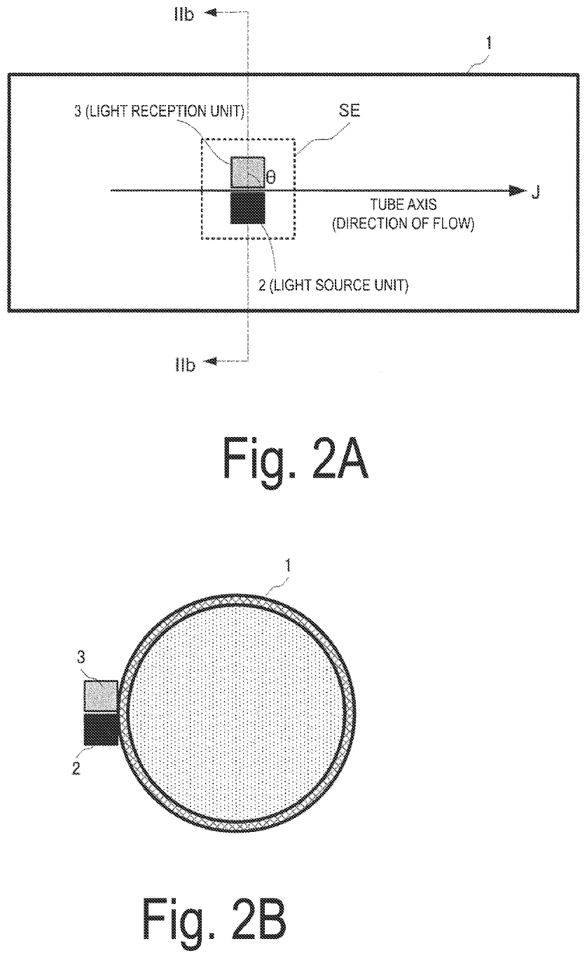

In the present embodiment, the light source unit 2 and the light reception unit 3 in each of the sensor elements SE (SE₁ to SE₃) are arranged in proximity to a direction intersecting a tube axis J of the tube 1 at a predetermined angle θ as viewed from a circumferential surface side of the tube 1 as illustrated in FIGS. 2A and 2B. FIG. 2A is a diagram viewed from the circumferential surface side of the tube 1, and FIG. 2B is a cross-sectional view taken along a line IIb-IIb of FIG. 2A. Although the angle θ is set to 90° in this example, the angle is not limited to 90°, and is preferably an angle within a range of 70° to 110° (90°±20°).

In the present embodiment, the sensor elements SE₁ to SE₃ are arranged such that light which is emitted from the light source unit 2 of the sensor element SE₁ and is transmitted through a fluid flowing through the tube 1 is received by the light reception unit 3 of the sensor element SE₂. Further, the sensor elements are arranged such that light which is emitted from the light source unit 2 of the sensor element SE₂ and is transmitted through the fluid flowing through the tube 1 is received by the light reception unit 3 of the sensor element SE₃. Furthermore, the sensor elements are arranged such that light which is emitted from the light source unit 2 of the sensor element SE₃ and is transmitted through the fluid flowing through the tube 1 is received by the light reception unit 3 of the sensor element SE₁.

A surface-emitting laser element (LD) in a near infrared region is mounted, as a light source, on the light source unit 2. In this case, a stable laser element with little output variation is preferably used as the light source, but an output of the laser element may be monitored and corrected. Further, a photodiode (PD) is provided, as the light reception unit 3, adjacent to the light source unit 2 at an interval of approximately 1 to 2 mm, and the light source unit 2 and the light reception unit 3 constitute the integrated sensor element SE.

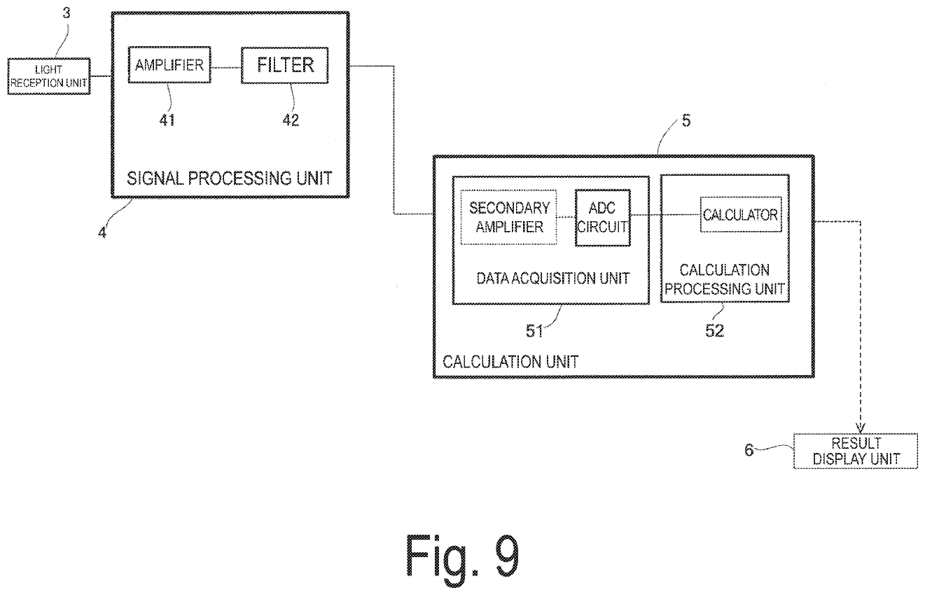

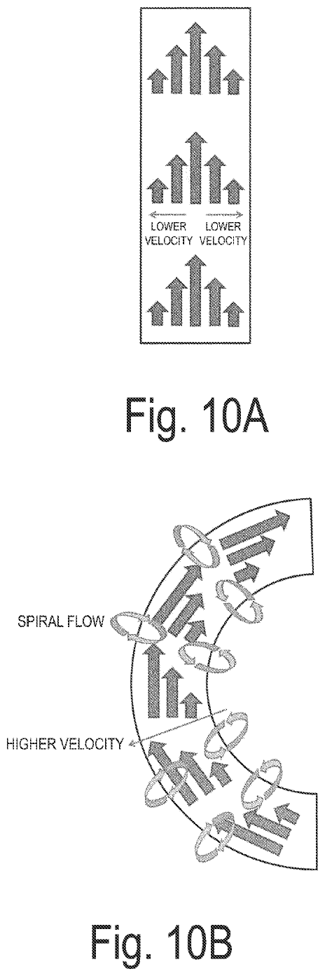

The sensor element SE is mounted on a printed circuit board that includes a signal processing unit 4. The sensor element SE mounted to the printed circuit board is referred to as a sensor head. A functional block diagram of the signal processing unit 4 is similar to the functional block diagram of FIG. 9. A calculation unit 7 is provided at a subsequent stage of the signal processing units 4₁ to 4₃ provided for the sensor elements SE₁ to SE₃. The calculation unit 7 includes a data acquisition unit 71 such as an analog-to-digital conversion circuit (ADC circuit), and a calculation processing unit 72 that performs a fast Fourier transform (FFT) or the like by using a calculator or the like.

A component arrangement of the signal processing units 4₁ to 4₃ may be appropriately omitted and changed depending on a measurement situation like a case where a filter in each of the signal processing units 4₁ to 4₃ is moved to the calculation unit 7. For example, the signal processing units 4₁ to 4₃ may be provided as one signal processing unit at a previous stage of the calculation unit 7.

In the fluid measurement device 101, the light emitted from the light source unit 2 of any one of the sensor elements SE is received by the light reception unit 3 of the adjacent sensor element SE. For example, the light source unit 2 of the sensor element SE₁ irradiates the fluid flowing through the tube 1 which serves as a flow path with light-source light having coherency (coherent light). The scatterers S that scatter the light-source light are contained in the fluid. Vinyl chloride is transparent, and has transmittance to light-source light wavelengths. When the light-source light is scattered by the scatterers S, a part of the light-source light is received by the light reception unit 3 of the sensor element SE₂. When a concentration of the scatterers S is low, the majority of the scattering is single scattering. However, as the concentration increases, the light-source light reaches the light reception unit 3 of the sensor element SE₂ by performing scattering multiple times. Transmitted light that is not scattered and reflected and scattered light from a stationary tube wall are also received as well.

The light received by the light reception unit 3 of the sensor element SE₂ is converted into an electrical signal. However, a beat signal is generated between light of which a frequency is changed due to Doppler shift and light of which a frequency is not changed (is changed very little), and is detected as an alternating current component. The electrical signal output by the light reception unit 3 of the sensor element SE₂ is typically weak. An output current is the order of about μA. Thus, the output current is amplified by using an amplification circuit such as a transimpedance amplifier arranged on the signal processing unit 4₂, and is then converted into a voltage signal having a level of, for example, approximately 1V which is easy to handle. Subsequently, the amplified signal is split, and only high-frequency (alternating current) components are extracted by causing one signal to pass through a high-pass filter. An appropriate value of approximately 1 to 100 Hz can be selected as a cutoff frequency of the high-pass filter.

After the signal which does not pass through the filter is converted into a digital signal by the ADC circuit in the data acquisition unit 71 in the subsequent calculation unit 7, high-frequency components are averaged by performing time average and are extracted as a direct current component, and is used for signal standardization or the like. This direct current component changes depending on a transmittance of a liquid, that is, the concentration of the scatterers S in the liquid, and thus, the change in the direct current component excluding the variation in the output of the laser element gives concentration information of the scatterers S. Accordingly, a calibration table is created by measuring a correspondence between a concentration of a target to be measured, a direct current component, and a flow velocity correlation feature value to be described below in the tube to be used in advance, and thus, the concentration of the scatterers S can be corrected for the flow velocity correlation feature value by using the direct current component obtained by subtracting the output variation of the laser element.

The high-frequency component typically has a value smaller than the direct current component by about single digit or double digits, and thus, the high-frequency component unnecessary for signal processing is removed by the low-pass filter after the high-frequency component is further amplified to a value appropriate for signal processing by a secondary amplifier, and is sent to the calculation unit 7. A cutoff frequency of the low-pass filter varies depending on the flow velocity of the scatterers S, but may be, for example, 20 MHz.

In the calculation unit 7, the high-frequency component from the signal processing unit 4₂ is converted into a digital signal by the ADC circuit in the data acquisition unit 71. The high-frequency component converted to the digital signal is sent to the calculation processing unit 72. The calculation processing unit 72 obtains a power spectrum by performing Fourier transform on the high-frequency component by FFT and calculating a power of the high-frequency component. When the power spectrum is obtained, the sum of products of a power P and a frequency f is calculated over a predetermined frequency range by Equation (1) represented below, and is used as a flow velocity correlation feature value ν.

Equation (1)

N=Σ(P(fi)×f) (1)

As described above, it has been described that the light from the light source unit 2 of the sensor element SE, is received by the light reception unit 3 of the adjacent sensor element SE₂ through the fluid flowing through the tube 1. However, even when the light from the light source unit 2 of the sensor element SE₂ is received by the light reception unit 3 of the adjacent sensor element SE₃ through the fluid flowing through the tube 1 and when the light from the light source unit 2 of the sensor element SE₃ is received by the light reception unit 3 of the adjacent sensor element SE₁ through the fluid flowing through the tube 1, the flow velocity correlation feature values ν are obtained as well.

The calculation processing unit 72 achieves fluid measurement by adding a calculation of multiplying three flow velocity correlation feature values ν by a calibration coefficient, calculating, for example, a value of the average flow rate from the three flow velocity correlation feature values ν to which this calculation is applied, and sending the calculated value of the average flow rate to a result display unit 6. A correction calculation of correcting frequency characteristics of the amplification and filter circuits can be appropriately performed in calculating the flow velocity correlation feature values ν. The ADC and the calculation processing are appropriately designed, and thus, the correction calculation or the like corresponding to an intensity of incident light and a degree of reflection using the direct current component or the like can be performed.

In the present embodiment, the integrated sensor elements SE are arranged around the tube 1 at equiangular intervals, and the light emitted from the light source unit 2 of any one sensor element SE is received by the light reception unit 3 of the adjacent sensor element SE through fluid flowing through the tube 1. Specifically, the scattered light is received by using the light reception unit 3 of the adjacent sensor element SE. Accordingly, the scattered light can be received from a fluid region of a range wider than in the related art, and the velocity distribution can be averaged over a wider region. The received signals of the plurality of sensor elements SE are averaged, and thus, the fluid measurement can be performed.

As a result, in the present embodiment, the influence of the change in the velocity distribution caused by the bending of the tube 1 can be reduced by 12% or more than in the related art. At this time, a transmitted light detection arrangement capable of selectively receiving forward-scattered light having a strong scattering intensity is used, and thus, an optical path becomes larger than in the related art. Accordingly, the influence of attenuation of the scattered light is canceled, and thus, a scattering signal having a magnitude approximately identical to a magnitude in the related art can be received.

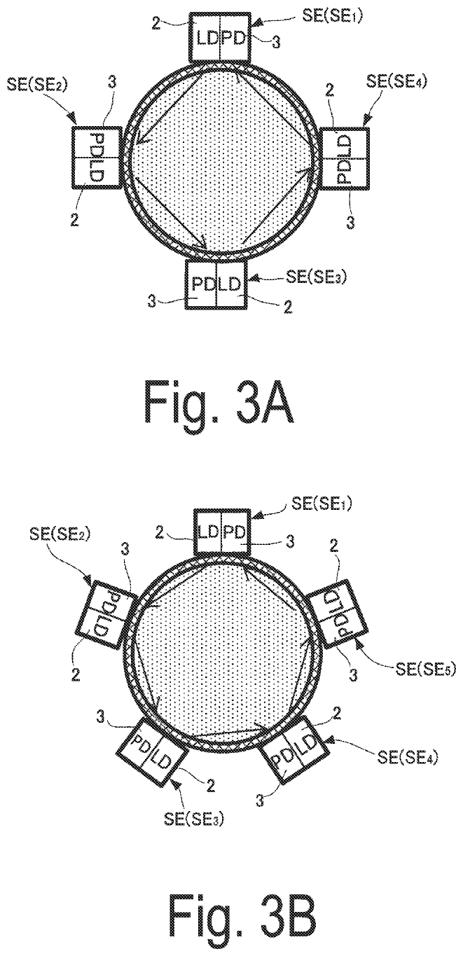

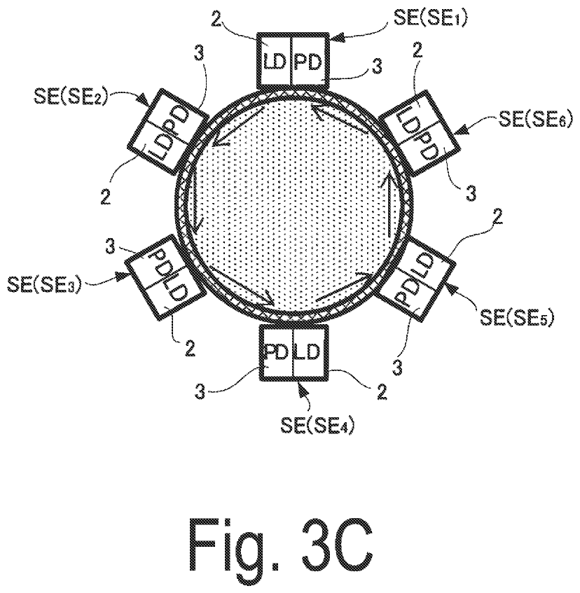

In this embodiment, the three sensor elements SE (three sensors) are arranged around the tube 1 at equiangular intervals. As illustrated in FIG. 3A, four sensors may be arranged. As illustrated in FIG. 3B, five sensors may be arranged. As illustrated in FIG. 3C, six sensors may be arranged. In this case, the influence of the change in the velocity distribution caused by the bending of the tube 1 can be reduced by using four sensors by 14% or more, by using five sensors by 15% or more, and by using six sensors by 16% or more than in the related art.

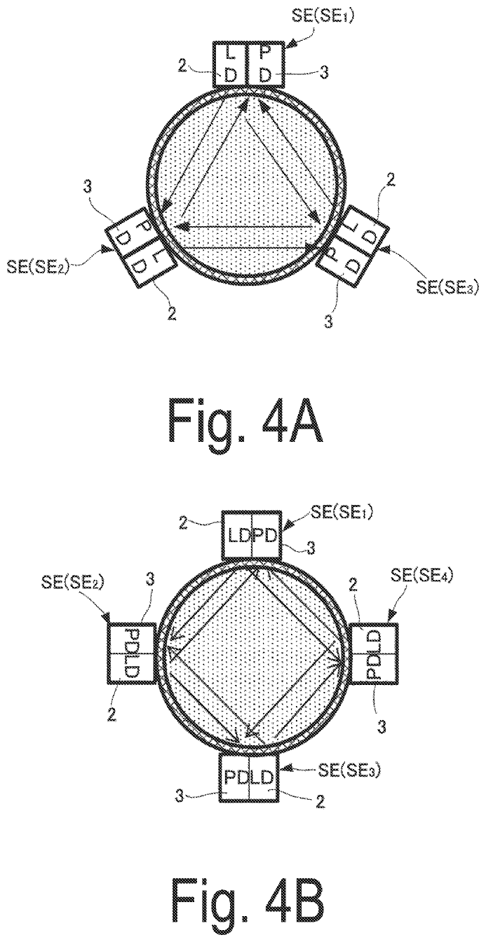

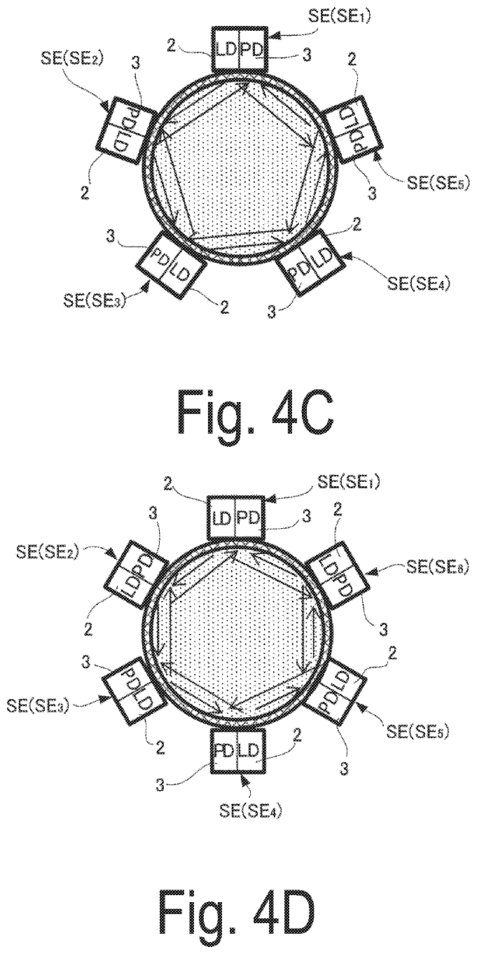

In this embodiment, the light from the light source unit 2 of the sensor element SE₁ is received by the light reception unit 3 of the sensor element SE₂. The light from the light source unit 2 of the sensor element SE₂ is received by the light reception unit 3 of the sensor element SE₃. The light from the light source unit 2 of the sensor element SE₃ is received by the light reception unit 3 of the sensor element SE₁. Meanwhile, as illustrated in FIG. 4A, the light rays from the light source units 2 of the sensor elements SE₁ and SE₃ may be received by the light reception unit 3 of the sensor element SE₂. The light rays from the light source units 2 of the sensor elements SE₁ and SE₂ may be received by the light reception unit 3 of the sensor elements SE₃. The light rays from the light source units 2 of the sensor elements SE₂ and SE₃ may be received by the light reception unit 3 of the sensor element SE₁. The same applies to cases where four sensors, five sensors, and six sensors illustrated in FIGS. 3A, 3B, and 3C are used (see FIGS. 4B, 4C, and 4D).

### Second Embodiment

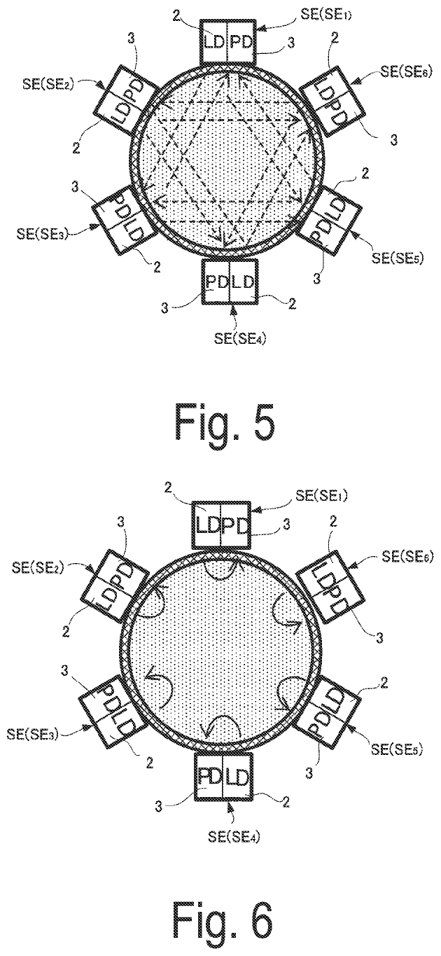

FIG. 5 illustrates a cross-sectional view of an element arrangement of a fluid measurement device according to a second embodiment of the present disclosure.

In the second embodiment, six integrated sensor elements SE (SE₁ to SE₆) are arranged around a tube 1 at equiangular intervals (intervals of 60°).

In the second embodiment, the scattered light is received by not the adjacent sensor element (first adjacent element) SE but the sensor element adjacent to the adjacent sensor element SE, that is, the sensor element (second adjacent element) SE adjacent with the sensor element SE interposed between these sensor elements.

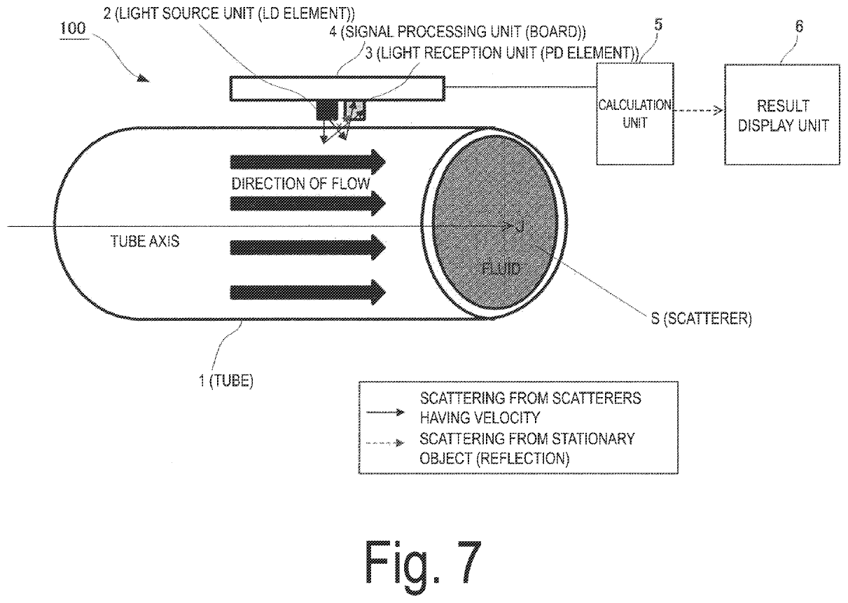

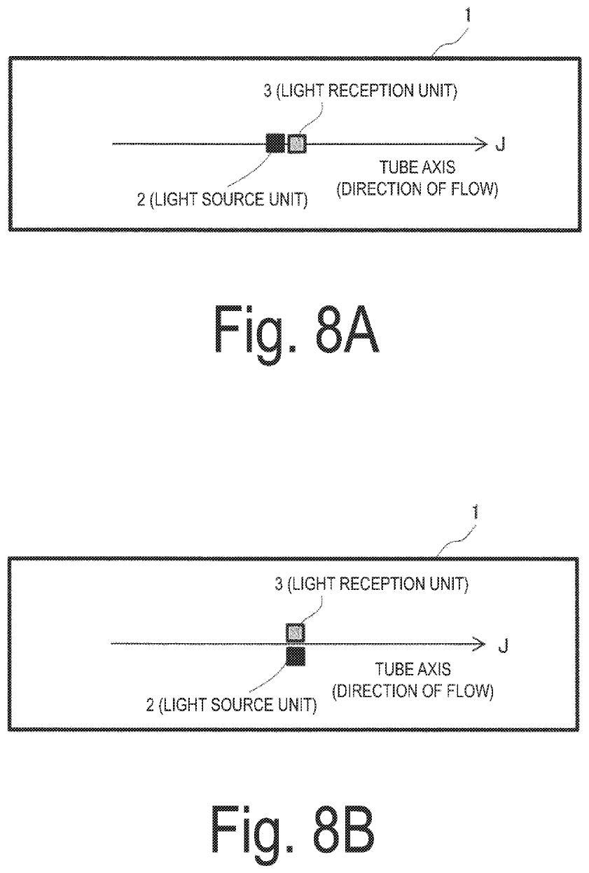

In the second embodiment, as illustrated in FIG. 6, the flow rate or the flow velocity is calculated by receiving the back-scattered light by using the light reception unit 3 in the identical sensor element SE and averaging the received signals together with a received signal of the back-scattered light.

In this manner, the influence of the change in the velocity distribution caused by the bending of the tube 1 can be reduced by 18% or more than in the related art.

It has been described in the second embodiment that the scattered light is received by using the second adjacent element and the received signal of the back-scattered light is also used to calculate the flow rate or the flow velocity. Alternatively, the scattered light may be received by using the first adjacent element, and a signal of this scattered light may also be used to calculate the flow rate or the flow velocity. In this case, the influence of the change in the velocity distribution caused by the bending of the tube 1 can be reduced by 20% or more than in the related art.

### Third Embodiment

A fluid measurement device according to a third embodiment of the present disclosure has an arrangement and measurement configuration similar to the arrangement and measurement configuration of the second embodiment, but focuses on the fact that the direct current component corresponding to the transmitted light increases or decreases depending on the concentration of the scatterers S having light absorption characteristics as described above.

Specifically, after the calibration table is created by measuring the correspondence between the concentration of the target to be measured, the direct current component, and the flow velocity correlation feature value in the tube to be used in advance, the calculation unit 7 corrects the flow velocity correlation feature value caused by the change in the concentration of the scatterers S by comparing the direct current component obtained by subtracting the output variation of the laser element with the calibration table. Accordingly, the flow velocity correlation feature value, for example, the average flow rate of the fluid in the tube that does not depend on the concentration of the scatterers S can be measured.

In this manner, in the third embodiment, the influence of the change in the velocity distribution caused by the bending of the tube 1 can be reduced by 18% or more than in the related art as in the second embodiment. The variation in the value of the flow rate dependent on the concentration of the scatterers S can be reduced by 15% or more. Accordingly, the variation in the value of the flow rate can be reduced by a total of 22% or more as the effect of reducing the variation in the value of the flow rate.

### EXPANSION OF THE EMBODIMENTS

The present disclosure has been described above with reference to the embodiments, but the present disclosure is not limited to the above-described embodiments. Various changes that can be understood by a person skilled in the art within the scope of the present disclosure can be made to the configuration and details of the present disclosure.

### REFERENCE SIGNS LIST

1 . . . Tube, 2 . . . Light source unit, 3 . . . Light reception unit, 4 (4₁ to 4₃) . . . Signal processing unit, 41 . . . Amplifier, 42 . . . Filter, 6 . . . Result display unit, 7 . . . Calculation unit, 71 . . . Data acquisition unit, 72 . . . Calculation processing unit, SE (SE₁ to SE₆) . . . Sensor element, S . . . Scatterer, 101 . . . Fluid measurement device

1.-7. (canceled)

8. A fluid measurement device, comprising:
first to N-th sensor elements arranged around a tube configured to allow a fluid containing scatterers to flow, wherein N is an integer of three or more, and wherein each of the first to N-th sensor elements includes a light source configured to irradiate the fluid with coherent light and a light receiver configured to receive and photoelectrically convert the coherent light;
a signal processor configured to amplify and filter signals which are received and photoelectrically converted by the light receivers of the first to N-th sensor elements; and
a calculator configured to convert the signals processed by the signal processor into digital signals, and calculate a flow velocity or a flow rate of the fluid based on the digital signals;
wherein the coherent light which is emitted from the light source of a first sensor element of the first to N-th sensor elements and is transmitted through the fluid flowing through the tube is received by the light receiver of a second predetermined sensor element of the first to N-th sensor elements; and
wherein, when a distance between the light source and the light receiver of the first sensor element is d and an outer radius of the tube is r, a distance between the first sensor element and the second predetermined sensor element is set to πd/2 or more and √3r or less.

9. The fluid measurement device according to claim 8, wherein the first to N-th sensor elements are arranged around the tube at equiangular intervals.

10. The fluid measurement device according to claim 8, wherein the second predetermined sensor element is a sensor element adjacent to the first sensor element.

11. The fluid measurement device according to claim 8, wherein:
N is an integer of 6 or more; and
the second predetermined sensor element is a sensor element adjacent to the first sensor element with a third sensor element adjacent to the first sensor element interposed between the second predetermined sensor element and the first sensor element.

12. The fluid measurement device according to claim 8, wherein the calculator is configured to calculate the flow velocity or the flow rate of the fluid based on a scattered light, which is a light scattered by the scatterers, the light configured to be emitted from the light source of the first sensor element and received by the light receiver of the first sensor element in addition to a light which is emitted from the light source of the first sensor element, transmitted through the fluid allowed to flow through the tube, and received by the light receiver of the second predetermined sensor element.

13. The fluid measurement device according to claim 8, wherein the calculator is configured to calculate the flow velocity or the flow rate of the fluid based on an average value of the signals received by the light receivers of the first to N-th sensor elements.

14. The fluid measurement device according to claim 8, wherein the calculator is configured to calculate concentration information of the fluid based on signals of transmitted light detected by the light receivers of the first to N-th sensor elements, and correct at least one value of the calculated flow velocity or the flow rate of the fluid based on the concentration information.

15. A method of measuring a fluid, the method comprising:
arranging first to N-th sensor elements around a tube that allows a fluid containing scatterers to flow, wherein N is an integer of three or more, and wherein each of the first to N-th sensor elements includes a light source to irradiate the fluid with coherent light and a light receiver to receive and photoelectrically convert the coherent light;
amplifying and filtering signals which are received and photoelectrically converted by the light receivers of the first to N-th sensor elements; and
converting the signals processed into digital signals and calculating a flow velocity or a flow rate of the fluid based on the digital signals;
wherein the coherent light which is emitted from the light source of a first sensor element of the first to N-th sensor elements and is transmitted through the fluid flowing through the tube is received by the light receiver of a second predetermined sensor element of the first to N-th sensor elements; and
wherein, when a distance between the light source and the light receiver of the first sensor element is d and an outer radius of the tube is r, a distance between the first sensor element and the second predetermined sensor element is set to πd/2 or more and √3r or less.

16. The method according to claim 15, wherein arranging the first to N-th sensor elements around the tube comprises arranging the first to N-th sensor elements around the tube at equiangular intervals.

17. The method according to claim 15, wherein the second predetermined sensor element is a sensor element adjacent to the first sensor element.

18. The method according to claim 15, wherein:
N is an integer of 6 or more; and
the second predetermined sensor element is a sensor element adjacent to the first sensor element with a third sensor element adjacent to the first sensor element interposed between the second predetermined sensor element and the first sensor element.

19. The method according to claim 15, wherein calculating the flow velocity or the flow rate of the fluid based on the digital signals comprises calculating the flow velocity or the flow rate of the fluid based on a scattered light, which is a light scattered by the scatterers, the light being emitted from the light source of the first sensor element and received by the light receiver of the first sensor element in addition to a light which is emitted from the light source of the first sensor element, transmitted through the fluid allowed to flow through the tube, and received by the light receiver of the second predetermined sensor element.

20. The method according to claim 15, wherein calculating the flow velocity or the flow rate of the fluid based on the digital signals comprises calculating the flow velocity or the flow rate of the fluid based on an average value of the signals received by the light receivers of the first to N-th sensor elements.

21. The method according to claim 15, further comprising:
calculating concentration information of the fluid based on signals of transmitted light detected by the light receivers of the first to N-th sensor elements; and
correcting at least one value of the calculated flow velocity or the flow rate of the fluid based on the concentration information.
